(19) United States
(12) Patent Application Publication
(10) Pub. No.: US 2011/0091566 A1
(43) Pub. Date: Apr. 21, 2011

(54) CONTROLLED RELEASE PHARMACEUTICAL COMPOSITIONS

(76) Inventor: Nirmal Mulye, Kendall Park, NJ (US)

(21) Appl. No.: 12/679,397

(22) PCT Filed: Sep. 25, 2008
(86) PCT No.: PCT/US08/77694

§ 371 Date: Dec. 16, 2010

Related U.S. Application Data
(60) Provisional application No. 60/995,197, filed on Sep. 25, 2007.

(51) Int. Cl. A61K 9/14; A61K 31/55; A61K 31/196; A61K 31/138; A61P 25/08; A61P 1/00; A61P 9/06; B29B 9/00; B29B 9/16

(52) U.S. Cl. 424/501; 514/217; 514/567; 514/652; 264/13; 264/6

(57) ABSTRACT

The present application discloses a sustained release composition in pellet form, wherein the core of the pellet comprises: (a) a therapeutically effective amount of a medicament; (b) 0.5 to 50% by weight of a water-soluble polymer; and (c) 25% by weight of a water-insoluble polymer applied as an aqueous latex dispersion and subsequently the water is removed, wherein the sum of the percentages of the medicament, the water-insoluble polymer and the water-soluble polymer is equal to or less than 100%. It also discloses methods of making this composition.

### FIELD OF THE INVENTION

The present invention relates to a sustained release pharmaceutical composition and to a method of using and preparing the same in order to control the rate of release and the site of release of the pharmaceutical composition.

### BACKGROUND OF THE INVENTION

It is desirable in the treatment of a number of diseases, both therapeutically and prophylactically, to provide active pharmaceutical ingredients in a sustained release form. The sustained release form provides a controlled rate of release of a medicament over an extended period.

Many controlled release compositions release the drug outside of the stomach. Some of the reasons for effecting release of the drug outside of the stomach are listed hereinbelow:

(1) prevention of decomposition of drugs that are unstable at pH values lower than a certain level;

(2) prevention of side effects brought about by the release of drugs in the stomach including irritation of the stomach wall by the drug;

(3) prevention of dilution of drug concentration in the intestines, attributable to disintegration of drugs in the stomach and their subsequent movement to the intestines;

(4) prolonged effect; and

(5) the drug affects areas outside of the stomach.

It is well known that well absorbed sustained release therapeutic drug dosage forms provide many advantages over conventional release dosage forms. The advantages include less frequent dosing of a medicament and resultant patient compliance, a more sustained drug blood level response, therapeutic action with less ingested drug and the mitigation of side effects. By providing a slow and steady release of the medicament over time, absorbed concentration spikes are mitigated or even eliminated by effecting smoother and more sustained blood level response.

Some sustained release pharmaceutical formulations are prepared such that they have a core containing the medicament or drug, which is surrounded by a coating which controls the release of the drug or medicament.

There are several reasons why the drug is not placed in the coating of these drug formulations. For example, the volume in the coatings is much smaller than if the volume of the underlying pellets so that more drug can be loaded onto the pharmaceutical composition if the drug is present in the core. Further, the coating may be needed for achieving controlled release. In addition, adjuvants, such as microcrystalline cellulose, starch and sugar are needed to form spheroids. Such adjuvants are hydrophilic and can cause disintegration leading to rapid release of a drug. Once again, the drug release in such cases is controlled by coating the pellets.

U.S. Pat. No. 4,867,985 describes formulation of controlled release pellets using high concentration of microcrystalline cellulose where the problem of disintegration caused by high amounts of microcrystalline cellulose has been alleviated by use of cellulose polymers. Although the disintegration is controlled by addition of cellulose polymers, such water-soluble polymers often fail to control the release over a very long time span.

U.S. Pat. Nos. 5,912,013 and 5,326,570 describe pellet formulation of the water insoluble drug carbamazepine, wherein a coating is utilized for achieving controlled release.

Known controlled release formulations rely on layered formulations wherein coatings cover a medicament. For instance, U.S. Pat. No. 5,202,128 to Morella, et al. discloses a sustained release pharmaceutical pellet composition for administration to a patient which comprises a core element containing a therapeutically effective amount of at least one active ingredient having an aqueous solubility of at least 1 in 30 and a coating on said core element which comprises:

(a) at least 35% by weight of a matrix polymer which is insoluble at a pH of from 1 to 7.5 and contributes to the control of the rate of release of the active ingredient in the stomach and intestines;

(b) from 1 to 30% of an enteric polymer which is substantially insoluble at a pH of from 1 to 4 sufficient to delay the release of the active ingredient in the stomach, but which is soluble at a pH of from 6 to 7.5 so as not to substantially delay release in the intestines; and

(c) from 1 to 60% of a compound soluble at a pH of from 1 to 4, sufficient to enable initiation of release of the active ingredient in the stomach, said percentages being by weight based upon the total weight of components (a), (b) and (c); the ratio of the components (a), (b) and (c) in said coating being effective to allow the initiation of the release of the active ingredient in the stomach at a slow rate and to control the release in the intestines at a rate faster than that in the stomach such that a dose of the pellet composition delivers to the patient a therapeutically effective amount of the active ingredient over the course of said predetermined interval.

U.S. Pat. No. 5,202,128 discloses a coating, which requires an insoluble polymer, and an enteric polymer and a compound soluble at a pH from 1 to 4. A coating comprised of the first two components alone prepared in accordance with the procedure described in U.S. Pat. No. 5,202,128 is so rigid and so strong that the coat will not dissolve, disperse or disintegrate sufficiently rapidly to release the medicament in effective concentrations while the pharmaceutical composition is in the gastrointestinal tract of the patient. The third component is thus required to initiate release of the drug in the stomach, that is, it helps to partially solubilize the coat so as to permit the initial release of the drug through the coat.

In the aforesaid paragraph, the release of the drug is controlled by components in the coating of the core. The present inventor has found that he can more easily control the release of the medicament and obtain a more uniform release of the medicament when the sustained release components are present in the core of the pellet.

### SUMMARY OF THE INVENTION

One aspect of the invention relates to a controlled release formulation in pellet form wherein the core comprises:

(a) a therapeutically effective amount of a medicament;

(b) 0.5 to 50% by weight of a sustained release water soluble polymer; and

(c) 1 to 25% by weight of a water insoluble polymer which was applied as an aqueous latex dispersion and subsequently the water is removed; and wherein the sum of the percentages of the medicament, the water insoluble polymer and the water soluble polymer is equal to or less than 100%.

Another aspect of the invention relates to a method of preparing a sustained release pharmaceutical composition in a pellet form, comprising:

(a) blending a therapeutically effective amount of a medicament and 0.5 to 50% by weight of a water soluble polymer;

(b) adding a granulating liquid to form a wet mass, wherein the granulating liquid comprises 1 to 25% by weight of a water insoluble polymer, which was applied as an aqueous latex dispersion and the water subsequently removed, and wherein the sum of the percentages of the medicament, the water insoluble polymer and the water soluble polymer is equal to or less than 100%;

(c) passing the wet mass through a perforated screen to obtain an extrudate; and

(d) forming spheres from the extrudate.

Another aspect of the invention relates to a method of preparing a sustained release pharmaceutical composition comprising:

(a) blending a therapeutically effective amount of a medicament and 0.5 to 50% by weight of a water-soluble polymer;

(b) adding a granulating liquid to form a wet mass, wherein the granulating liquid comprises 1 to 25% by weight of a water insoluble polymer in an aqueous latex dispersion, and wherein the sum of the percentages of the medicament, the water insoluble polymer and the water soluble polymer is equal to or less than 100%; and

(c) placing the wet mass from step (b) onto a rotor processor to form spheres wherein the granulating liquid is sprayed onto the blend.

Another aspect of the invention relates to a method of treating a disease in a patient requiring a sustained release formulation of a medicament for treating said disease, said treatment comprising administering to the patient an effective amount of the pharmaceutical composition of the present invention.

### DETAILED DESCRIPTION OF THE INVENTION

By mammal, it is meant a vertebrae of the class Mammalia that is characterized by possession of hair and mammary glands. Examples include, inter alia, dog, cat, horse, pig, goat, cow, and human being. The preferred species of mammal to which the sustained release formulation of the present invention is to be administered is human.

The terms “sustained release” and “controlled release” are being used interchangeably. As used herein, they refer to the release of the active ingredient at such a rate that blood levels are maintained within the therapeutic range but below toxic levels over an extended period of time, e.g., 4 to 24 hours or even longer.

The term “bioavailability” as used herein refers to the rate and extent to which the active drug ingredient is absorbed from the pharmaceutical formulation and is available at the site of drug action.

One aspect of the invention relates to a sustained release pharmaceutical composition in pellet form, in which the core of the pellet comprises:

(a) a therapeutically effective amount of a medicament;

(b) about 0.5 to about 50% by weight of a water soluble polymer; and

(c) about 1 to about 25% by weight of a water insoluble polymer applied as an aqueous latex dispersion and subsequently the water is removed, wherein the sum of the percentages of the medicament, the water insoluble polymer and the water-soluble polymer is equal to or less than 100%,and wherein said composition is in the form of a granulated medium.

The insoluble polymer is any suitable pharmaceutically acceptable non-toxic polymer substantially insoluble in aqueous media, e.g., water, independent of the pH thereof. Thus, it is insoluble in the gastric fluid, i.e., at pH's less than 4 and is insoluble in the intestinal fluid, at pH's between 6.0 and 7.5 and at the various pH's between 4 and 6 at 25° C. It is also insoluble in water at pH's greater than 7.5 at 25° C. The polymer may be a cellulose ether, cellulose ester or cellulose ether-ester in which a part or all of the hydroxyl groups on the cellulose skeleton is substituted. In view of the requirement that the insoluble polymer is substantially insoluble in both the gastric and intestinal fluids, these cellulose derivatives having a minimal number of hydrophilic substituents are preferred. Examples include ethyl cellulose, acetyl cellulose, nitrocellulose, and the like.

Other examples of insoluble polymers include lacquer, and acrylic and/or methacrylic ester polymers, polymers or copolymers of acrylates or methacrylates having a low quaternary ammonium content, or mixtures thereof and the like. Other examples include methylmethacrylate polymers e.g., EUDRAGIT RS® which is a water-insoluble film former based on neutral swellable methacrylic acid esters with a small proportion of trimethylaminoethyl methacrylate chlorides, the molar ratio of the quaternary ammonium groups to the neutral ester groups is 1:40 (.about.25 meq/100 g), EUDRAGIT RL® which is also a water insoluble, swellable film former based on neutral methacrylic acid esters with a small proportion of trimethylaminoethyl methacrylate chloride, the molar ratio of quaternary ammonium groups to the neutral ester group is 1:20 (corresponding to about 50 meq/100 g), EUDRAGIT NE®, which is a neutral methacrylic acid ester without any functional groups that forms a water insoluble film, and the like. The water insoluble polymers are preferably cellulose derivatives selected from the group consisting of ethyl cellulose, cellulose acetate, and methylmethacrylate copolymers. Most preferably, the water insoluble polymer is ethyl cellulose.

The most preferred water insoluble polymers are ethyl cellulose and cellulose acetate and methylmethacrylate polymers. The most preferred water insoluble polymer is ethyl cellulose.

Non-limiting examples of the aqueous latex dispersion used in the present composition is selected from the group consisting of ethyl cellulose water dispersion, cellulose acetate aqueous dispersion, methyl methacrylate aqueous dispersion, and cellulose ether.

The water insoluble polymer is present in the core in an amount ranging from about 1 to about 25% by weight of the pellet. It is more preferred that it comprises at least about 3 to about 15% by weight of the pellet, and most preferably from about 5% to about 15% by weight of the pellet.

It is to be understood that when the water insoluble polymer is mixed in the composition, it is applied as an aqueous dispersion. After it is added to the composition comprising the drug and/or water soluble polymer, the water present in the emulsion is removed by conventional means, such as by drying, evaporation and the like. The weight indicated hereinabove refers to the weight of the water insoluble polymer and excludes any water and any other component that may present in the aqueous dispersion.

The water-soluble polymer is soluble or swellable in water. Preferably, it dissolves and/or swells in water at room temperatures. Examples include the cellulose ethers, hydrocolloid (gum), polyvinyl alcohol and polyvinyl pyrrolidone.

The cellulose ethers for use in this invention are hydrophilic polymers, which are commercially available. Examples of cellulose ethers used in the present invention include carboxymethyl cellulose, methyl cellulose, hydroxyethyl cellulose, hydroxypropyl cellulose, hydroxybutyl cellulose, hydroxyethylmethyl cellulose, hydroxyethylethyl cellulose, hydroxypropylmethyl cellulose, hydroxypropylethyl cellulose, hydroxybutylmethyl cellulose, hydroxybutylethyl cellulose, carboxymethyl cellulose and salts thereof.

Moreover, the present invention includes the use of hydroxypropylmethyl cellulose in its various forms.

Hydroxypropylmethyl cellulose is commercially available in various grades, under several tradenames, including METHOCEL, E, F, J and K from The Dow Chemical Co., USA, HPM from British Celanese Ltd. England and Metaluse SH from Shin Etsu, Ltd, Japan. The various grades available under a given tradename represent differences in methoxyl and hydroxypropoxyl content as well as molecular weight. The methoxyl content ranges from 16.5 to 30 weight % and the hydroxypropoxyl content ranges from 0 to 32 weight -%, as determined by the method described in ASTM D-2363-72. All of these various forms of hydroxypropylmethyl cellulose are contemplated to be used in the present invention. For example, the present invention contemplates the use of Methocel K in its various forms having a methoxyl content of 19-24% and a hydroxypropoxyl content of 7-12%, Methocel E in its various forms, having a methoxyl content of 28-30 to and a hydroxypropyl content of 7-12%, Methocel F in its various forms having a methoxyl content of 27-30% and a hydroxypropoxyl content of 4-7.5%, Methocel A in its various forms, having a methoxyl content of 27.5-31.5% and about 0% hydroxypropoxyl content.

Commercial designations of the various hydroxypropylmethyl cellulose are based on the viscosities of 2% aqueous solutions at 20° C. The viscosities range from 15 cps to 30,000 cps and represent number average molecular weights ranging from about 10,000 to over 150,000, as calculated from the data in the “Handbook of Methocel Cellulose Ether Products” (The Dow Chemical Co., 1974).

Examples of hydroxypropylmethyl cellulose include Metalose 60 5H50 which is a hydroxypropylmethyl cellulose having a hydroxypropoxyl content of 9-12 weight % and a number average molecular weight of less than 50,000; Methocel E4M, having a 28-30 weight % methoxyl content, a viscosity of 4000 cps, a hydroxy-propoxyl weight % of 7-12 and a number average molecular weight of 93,000; Methocel E10M, having a viscosity of 10,000 cps, a 28-30 weight % methoxyl content, 7-12 weight % hydroxypropoxyl, Methocel K4M, having a number average molecular weight of 89,000, viscosity of 4,000, 19-24% weight % methoxyl content, and a 7-12 weight % hydroxypropoxyl content; Methocel K15M, having a number average molecular weight of 124,000, a 19-24 weight % methoxyl content and a 7-12 weight % hydroxypropoxyl content; and K100M, having a viscosity of 100,000 cps and a 19-24 weight % methoxyl content and is 7-12 weight % hydroxypropoxyl content, Methocel J5M, J12M, J20M and J75M, having viscosities of 5,000, 12,000, 20,000, and 75,000, cps, respectively and the like. Various hydroxypropylmethyl cellulose materials, which can also be used in the present formulation, are described in U.S. Pat. No. 3,870,790 to Schorr, U.S. Pat. No. 4,226,849 to Schorr, U.S. Pat. No. 4,357,469 to Schorr, U.S. Pat. No. 4,369,172 to Schorr, et al., U.S. Pat. No. 4,389,393 to Schorr, et al., U.S. Pat. No. 4,259,314 to Lowey, U.S. Pat. No. 4,540,566 to Davis, et al., U.S. Pat. No. 4,556,678 to Hsiau, the contents of all of which are incorporated herein by reference. The present formulation may contain one cellulose ether or a combination of cellulose ethers.

Another water soluble polymer is the hydrocolloid. Examples include guar gum, alginic acid and its pharmaceutically acceptable salts e.g., sodium alginate and xanthan gum. The present formulation may contain one hydrocolloid or a combination of hydrocolloids.

Xanthan gum is a high molecular natural carbohydrate and more particularly polysaccharide produced by the fermentation process of the microorganism Xanthomonas campetris. The molecular weight of the xanthan gum polymer is probably on the order of 2 million, but has reported to be as high as 13-50 million. These reported differences are most probably due to association phenomena between the polymer chains. The xanthan gum used in the present invention is preferably in a dry, free, flowing granular or powdered form, with a preferred average particle size ranging between 850-74 microns.

The water-soluble polymers are preferably cellulose derivatives selected from the group consisting of hydroxypropyl methylcellulose (HPMC), hydroxypropyl cellulose (HPC), sodium carboxymethylcellulose (NaCMC), gums, polyvinyl alcohol (PVA) and polyvinylpyrrolidone (PVP). Preferably, the water soluble polymer is hydroxypropyl methylcellulose.

The weight percentage of the water-soluble polymer in the pellets preferably range from about 1 to about 20% by weight, more preferably from about 2 to about 10% by weight, and most preferably from about 3 to about 20% by weight of the composition.

The ratio of the water-soluble polymer to the water insoluble polymer preferably ranges from about 5:95 to about 50:50, preferably from about 5:95 to about 30:70, more preferably from about 5:95 to about 20:80.

The active ingredient can be of any type of medication which acts locally in the mouth or acts systemically, which in the case of the latter, can be administered orally, to transmit the active medicament into the gastrointestinal tract and into the blood, fluids and tissues of the body. The pharmaceutical composition of the present invention can contain one active ingredient or more than one active ingredient. The preferred pharmaceuticals are hydrophobic drugs. The medicament preferably has a limited water solubility of less than about 40 mg/ml, more preferably less than about 25 mg/ml, even more preferably less than about 1 mg/ml.

Representative active medicaments include antacids, anti-inflammatory substances, coronary vasodilators, cerebral vasodilators, peripheral vasodilators, anti-infectives, psychotropics, antimanics, stimulants, anti-histamines, laxatives, decongestants, vitamins, gastro-intestinal sedatives, antidarrheal preparations, anti-anginal drugs, vasodilators, antiarrythmics, anti-hypertensive drugs, vasoconstrictors drugs useful to treat migraines, anticoagulants and antithrombotic drugs, analgesics, anti-pyretics, hypnotics, sedatives, anti-emetics, anti-nauseants, anticonvulsants, neuromuscular drugs, hyper- and hypoglycemic agents, thyroid and antithyroid preparations, diuretics, antispasmodics, uterine relaxants, mineral and nutritional additives, antiobesity drugs, anabolic drugs, erythropoietic drugs, antiasthmatics, expectorants, cough suppressants, mucolytics, antiuricemic drugs and other drugs or substances acting locally in the mouth, such as topical analgesics, local anaesthetics, vitamins, and the like.

Vitamins include such vitamins as vitamin A, vitamin D, vitamin B (d-alpha-tocopherol acetate, etc.), vitamin B₁, (dibenzoylthiamin, fursultiamine hydrochloride, etc.), vitamin B₂ (riboflavin butyrate, etc.), vitamin B₆ (pyridoxine hydrochloride, etc.), vitamin C (ascorbic acid, sodium L-ascorbate, etc.), vitamin B₁₂ (hydroxocobalamin acetate, etc.); minerals such as calcium, magnesium, and iron.

Examples of the hydrophobic drugs which can be formulated in accordance with the present invention include the following:

Analgesics and anti-inflammatory agents: aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac.

Anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.

Anti-arrhythmic agents: amiodarone HCl, disopyramide, flecainide acetate, quinidine sulphate. Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.

Anti-coagulants: dicoumarol, dipyridamole, nicoumalone, phenindione.

Anti-depressants: amoxapine, maprotiline HCl, mianserin HCL, nortriptyline HCl, trazodone HCL, trimipramine maleate.

Anti-diabetics: acetohexamide, chlorpropamide, glibenclamide, gliclazide, glipizide, tolazamide, tolbutamide.

Anti-epileptics: beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid.

Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid.

Anti-gout agents: allopurinol, probenecid, sulphin-pyrazone.

Anti-hypertensive agents: amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL.

Anti-malarials: amodiaquine, chloroquine, chlorproguanil HCl, halofantrine HCl, mefloquine HCl, proguanil HCl, pyrimethamine, quinine sulphate.

Anti-migraine agents: dihydroergotamine mesylate, ergotamine tartrate, methysergide maleate, pizotifen maleate, sumatriptan succinate.

Anti-muscarinic agents: atropine, benzhexol HCl, biperiden, ethopropazine HCl, hyoscyamine, mepenzolate bromide, oxyphencylcimine HCl, tropicamide.

Anti-neoplastic agents and Immunosuppressants: aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone.

Anti-protazoal agents: benznidazole, clioquinol, decoquinate, diiodohydroxyquinoline, diloxanide furoate, dinitolmide, furzolidone, metronidazole, nimorazole, nitrofurazone, ornidazole, tinidazole.

Anti-thyroid agents: carbimazole, propylthiouracil.

Anxiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

.beta.-Blockers: acebutolol, alprenolol, atenolol, labetalol, metoprolol, nadolol, oxprenolol, pindolol, propranolol.

Cardiac Inotropic agents: amrinone, digitoxin, digoxin, enoximone, lanatoside C, medigoxin.

Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.

Diuretics: acetazolamide, amiloride, bendrofluazide, bumetanide, chlorothiazide, chlorthalidone, ethacrynic acid, frusemide, metolazone, spironolactone, triamterene.

Anti-parkinsonian agents: bromocriptine mesylate, lysuride maleate.

Gastro-intestinal agents: bisacodyl, cimetidine, cisapride, diphenoxylate HCl, domperidone, famotidine, loperamide, mesalazine, nizatidine, omeprazole, ondansetron HCl, ranitidine HC, sulphasalazine.

Histamine H-Receptor Antagonists: acrivastine, astemizole, cinnarizine, cyclizine, cyproheptadine HCl, dimenhydrinate, flunarizine HCl, loratadine, meclozine HC1, oxatomide, terfenadine.

Lipid regulating agents: bezafibrate, clofibrate, fenofibrate, gemfibrozil, probucol.

Nitrates and other anti-anginal agents: amyl nitrate, glyceryl trinitrate, isosorbide dinitrate, isosorbide mononitrate, pentaerythritol tetranitrate.

Nutritional agents: betacarotene, vitamin A, vitamin B₂, vitamin D, vitamin E, vitamin K.

Opioid analgesics: codeine, dextropropyoxyphene, diamorphine, dihydrocodeine, meptazinol, methadone, morphine, nalbuphine, pentazocine.

Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.

Stimulants: amphetamine, dexamphetamine, dexfenfluramine, fenfluramine, mazindol.

Mixtures of hydrophobic drugs may, of course, be used where therapeutically effective.

A preferred active ingredient is carbamazepine. Another preferred active ingredient is mesalamine. A further preferred active ingredient is propafenone. The active ingredient may also be present as the pharmaceutically acceptable salt of any of the drugs mentioned hereinabove.

The active ingredient can be present in any suitable pharmaceutically effective amount. The amount of active ingredient is dependent on the potency of the active ingredient and on the desired dosage strength and volume of a unit dose of the drug product. The active ingredient can be present in amounts of approximately 0.1 to 95% by weight, based on the total weight of the core element. However, utilizing the formulation described herein, the pellet is capable of containing a high concentration of drug. For example, in one embodiment, the drug is present in amounts greater than about 30% by weight of the pellet, and preferably greater than about 40% by weight and even more preferably greater than about 50% by weight of the composition. In another embodiment, the drug is present in more than about 60% by weight of the pellet. In another embodiment, the active ingredient is present in an amount ranging from about 40% to about 75% by weight of the pellet.

The ratio of active ingredient to the rest of the composition varies according to the unit dosage of drug to be employed and the size of the starting spheres. It is apparent that the ratio could vary widely depending on the dosage amount.

The core of the sustained release composition of the present invention optionally can include other conventional additives such as diluents, plasticizers, excipients, anti-oxidants, coloring agents, anti-oxidants, sweeteners, flavoring agents, preservatives and any other optional ingredients. The composition can be provided in unit dosage form, preferably in the form of a pellet.

In another embodiment, the composition of the invention further comprises a diluent ranging from about 5% to about 95% by weight of the total composition, preferably from about 10% to about 60% by weight of the total composition, more preferably from about 20% to about 40% by weight of the total composition.

Non-limiting examples of the diluent include microcrystalline cellulose, starch, and maltodextrin. Preferably, the diluent is microcrystalline cellulose or maltodextrin. More preferably, the diluent is microcrystalline cellulose, especially silicified microcrystalline cellulose.

In a preferred embodiment of the inventive composition, the water soluble polymer is present from about 0.5% to 50% by weight, the water insoluble polymer is present from about 1% to about 25% by weight, and microcrystalline cellulose is present from about 1% to about 65% by weight.

The composition of the core of the pellet of the present invention can also include a wetting agent or emulsifying agent or defoaming agent. Non-limiting examples include sodium lauryl sulfate, emulsifying wax, lecithin, polymethyl siloxane emulsion, cetostearyl alcohol, cetyl alcohol, glyceryl monostearate, sodium stearate, lanolin alcohols, polyoxyethylene sorbitan fatty acid esters, polyoxy ethylene stearates, propylene glycol alginate, stearyl alcohol, sorbitan esters and the like or a mixture thereof.

The composition of the invention can include a surfactant ranging from about 0.1 to about 10% by weight of the total composition, preferably from about 0.5 to about 2% by weight of the total composition. The surfactant is preferably sodium lauryl sulfate. Sodium lauryl sulfate can also be present in the amounts 0.1 to 5%, preferably 0.5 to 2% by weight of tee composition.

Other optional ingredients that are also typically used in pharmaceuticals may also be present in the core of the pellet, such as coloring agents, preservatives (e.g., methyl parabens), artificial sweeteners, flavorants, anti-oxidants, and the like. Artificial sweeteners include, but are not limited to, sodium saccharin, aspartame, dipotassium glycyrrhizinate, stevia, thaumatin and the like. Flavorants include, but are not limited to, lemon, lime, orange and menthol. The colorants include, but are not limited to, various food colors, e.g., FD&C colors, such as FD&C Yellow No. 6, FD&C Red No. 2, FD&C Blue No. 2, food lakes and the like. Examples of anti-oxidants include ascorbic acid, sodium metabisulphite and the like. These optional ingredients, if present, preferably are present in amounts ranging from about 0.1% to about 5% by weight of the core element and most preferably less than about 3% (w/w) of the core element.

The diameter of the spheres of the invention preferably ranges from about 0.2 mm to about 2.5 mm, preferably from about 0.5 mm to about 1.5 mm, more preferably from about 0.8 mm to about 1.2 mm.

As described herein, the medicament, the water-soluble polymer, and the water insoluble polymer and the optional ingredients described herein, are located in the inner core of the pellet. A coating may or may not be present on the core. In one embodiment, a coating is not present and the control of the release of the drug is controlled by the composition in the core. If a coating is present, it may be coated with one of the many readily available coating systems. Nevertheless, it is to be understood that the components described hereinabove; the water insoluble drug, the water insoluble polymer, and the water-soluble polymer are present in the core. The coating, if present, surrounds the core, and may contain additional water insoluble polymer(s) and water-soluble polymer(s) and the other optional ingredients described hereinabove. However, the release of the drug in the present composition, even if a coating is present, is controlled by the composition in the core even though the coating composition may modify slightly the release of the drug from the present composition, either by accelerating or slowing its release slightly. For example, the composition in the coating may increase or decrease the release of the drug by at most about 10% relative to the release of the drug from the core. Although the coating may contain the medicament, it is preferred that the medicament is present solely in the core of the pellet mostly by the ingredients in the core.

The coating may either be non-functional or functional. The coating may, for example, be coated with one of numerous polymeric film coatings frequently employed by formulation chemist. Examples include HPMC, carboxymethylcellulose, hydroxypropylcellulose, methylcellulose, ethylcellulose, acrylic resins, polyvinyl povidone (PVP), polyldivinyl diethylaminoacetate, cellulose acetate phthalate, polyvinyl acetate phthalate, acrylic latex emulsions, ethylcellulose latex emulsions, hydrocolloids, starch, and the like.

The coating may contain the optional ingredients described hereinabove. If present, the coat may be present in amounts ranging from about 1 to about 20% by weight and preferably from about 1 to about 15% of the composition.

The composition of the present invention may, if desired, contain a controlled release polymer coating, an enteric polymer coating, a water soluble polymer coating, or combinations thereof.

The composition of the invention can include optional coatings such as conventional additives like plasticizers, film forming materials, film forming agents, polymer particles, surfactants, coloring agents, excipients, including fillers, such as talc, titanium dioxide or barium sulphate or antioxidants in order to improve the properties of the preparation, as is well known to the skilled artisan.

The optional coating can also contain such lubricants as talc, calcium stearate, colloidal silicon dioxide, magnesium stearate, zinc stearate, aluminum stearate, polyethylene glycol, glycerin, and the like or a mixture thereof.

The uncoated compositions of the present invention are prepared by art recognized techniques.

The pellet cores of the present invention are prepared by various techniques known to the skilled artisan, e.g., high shear pelletization, fluid-bed pelletization, hot-melt and extrusion spheronization.

In one embodiment, the composition of the invention is formed by blending the medicament, the water-insoluble polymer, the water soluble polymer and the other optional ingredients to form the granulated medium. The components are thoroughly mixed until the mixture is substantially homogenous. The granulated medium can be placed on a spheronizer to produce spheres.

In another embodiment, the water-soluble polymer is added as a powder. In another embodiment, the water soluble polymer is dissolved in the aqueous latex dispersion of the water insoluble polymer. In yet another embodiment, the water soluble polymer is added as a powder as well as dissolved in a latex dispersion.

Another aspect of the invention relates to a method of preparing a sustained release pharmaceutical composition comprising:

(a) blending a therapeutically effective amount of a medicament and 0.5 to 50% by weight of a water soluble polymer;

(b) adding a granulating liquid to form a wet mass, wherein the granulating liquid comprises 1 to 25% by weight of a water insoluble polymer in an aqueous latex dispersion, and wherein the sum of the percentages of the medicament, the water insoluble polymer and the water soluble polymer is equal to or less than 100%;

(c) passing the wet mass through a perforated screen to obtain an extrudate; and

(d) forming spheres from the extrudate.

The extrudate in step (d) is preferably placed on a spinning plate to make spheres.

In another embodiment, the inventive method further comprises drying the spheres by fluid bed drying or tray drying, preferably by fluid bed drying.

Another aspect of the invention relates to a method of preparing a sustained release pharmaceutical composition comprising:

(a) blending a therapeutically effective amount of a medicament and 0.5 to 50% by weight of a water soluble polymer;

(b) adding a granulating liquid to form a wet mass, wherein the granulating liquid comprises 1 to 25% by weight of a water insoluble polymer in an aqueous latex dispersion, and wherein the sum of the percentages of the medicament, the water insoluble polymer and the water soluble polymer is equal to or less than 100%; and

(c) and placing the wet mass from step (b) onto a rotor processor to form spheres wherein the granulating liquid is sprayed onto the blend.

This method can further comprise drying the spheres by fluid bed drying or tray drying, and preferably fluid bed drying.

The water soluble polymer may be dissolved in the latex dispersion provided the water soluble polymer is present in low enough concentration. The water soluble polymers increase the viscosity of the latex dispersion and making it difficult to pour into the blend to achieve granulation. The latex dispersion may be diluted with water to achieve optimum volume of granulating medium.

In all of the embodiments described hereinabove, it is preferred that the active ingredient (i.e., the drug), that water-soluble polymer, the water insoluble polymer and the other ingredients that are to be present in the core are mixed together thoroughly, and then the resulting product is subjected to extrusion.

All of the components in the core are mixed together in a blend to form a homogenous and uniform mixture. The various components are substantially uniform throughout the mixture so that, after being made into a pellet, any section of the pellet will have approximately the same composition as any other section of the pellet.

To form the active core, any suitable apparatus can be used. They include a rotor granulator, pan coater, spheronizer and extruder.

In another aspect of the present invention, the core elements, including all of its components except the water insoluble polymer, are placed into a fluidized bed reactor or apparatus or rotary fluid bed machine. In this embodiment, the granulating liquid comprised of the water-insoluble polymer is applied by spraying it on the core elements composition comprising the active ingredient and the water-soluble polymer and the other ingredients in the core (except the water-insoluble polymer), which have been thoroughly mixed to form a substantially uniform mixture. Examples of a fluid bed bottom spray coater that can be used include, the Wurster coating apparatus (Pharmaceutical Pelletization Technology, (1989), pp. 50-54, ed. Isaac Ghebre-Sellassie, Marcel Dekker, Inc., New York and Basel).

The solution or suspension of active ingredient is formed by dissolving or by dispersion of the active ingredient in distilled water or organic solvents used in the art. The water-soluble polymer is thoroughly mixed therein. Other optional ingredients to be present in the core for example, antiadherents and/or other excipients or ingredients as is desirable or appropriate are added to the solution or suspension.

After the granulating liquid comprising the water insoluble polymer is applied, the components are thoroughly mixed, the active ingredient, and the components are subjected to an extrusion followed by marumerization to form the core element.

Spray coating of the core elements may be undertaken utilizing bottom, top or tangentially located spray nozzles. A bottom spray nozzle may reside proximate to the base of the fluidized bed facing upwards while a top spraying nozzle is located above the contents of the bed and facing downwards. The spray nozzle may reside in the mid-section of the fluidized bed and be oriented such as to spray tangentially to the rotating core elements.

The core elements formed by whatever method are then subjected to a drying step. The drying step may be conducted in a fluidized bed or drying oven.

The pharmaceutical composition can be dried by standard techniques. In the drying step, all or substantially all of the water in the coating composition is removed. For example, the pharmaceutical composition may be dried for about ¼ to about 2 hours at a temperature of from about 30° C. to about 60° C., preferably for about ½ hour at 40° C., and allowed to cool to ambient temperature, and if necessary, sieved through an appropriately sized mesh. Afterwards, the pharmaceutical composition of the present invention may be subjected to other conventional procedures, including polishing, or sugar coating.

Following formation and drying of the pellet, the coating composition, if necessary, of the present invention is applied using standard techniques known in the art.

Another aspect of the invention relates to a method of treating a disease in a patient requiring a sustained release formulation of a medicament for treating said disease, said treatment comprising administering to the patient an effective amount of the pharmaceutical composition of the invention.

By having the active drug and the water insoluble polymer and water soluble polymer, as described, present in the core, the present invention has several advantages over the pellets whereby the release of the drug is controlled by the composition in the coating or when the medicament is present in the coating. For example, by placing the medicament in the core, the composition of the present invention can hold more drug than if the active ingredient were in the coating. For instance, the present formulation can be comprised of at least about 30%, more preferably at least 40% and especially preferably greater than almost 50% and most preferably greater than about 60% by weight active ingredient.

Moreover, the present invention has a less variable controlled release profile than if the sustained release polymers were in the coating. The release profile of the drug from the pellets of the present invention is more uniform from batch to batch than if the water insoluble and water insoluble polymers were in the coating. Moreover, it is easier to manufacture the pellet in which the active ingredient and the water insoluble polymer and the water-soluble polymers are contained in the core, rather than in the coating.

In the sustained release pellet form of the present invention, there is significantly less fluctuations in plasma concentration of active ingredients at steady state over a 24 hour period, which may allow for less frequent dosing relative to the active ingredients in an uncoated form of a different type of dosage form. For example, if the present formulation were made into an oral tablet, not only would the release be more variable any release of the active component would be extremely, extremely slow.

The term unit dosage form as employed herein refers to physically discrete units suitable as unitary dosages to human subjects and other mammals, each unit containing a predetermined quantity of medicament calculated to produce the desired effect, in association with the other ingredients of the formulation disclosed herein.

In the formulations described hereinabove, the percent of the components are calculated on a dry weight basis, without reference to any water or other components present.

Unless indicated to the contrary, all percentages are weight percentages relative to the pharmaceutical composition.

Moreover, the terms “active ingredient”, “drug”, and “medicament” are used interchangeably.

The singular connotes the plural and vice versa.

These and other advantages of the present invention, as well as aspects of preferred embodiments, are illustrated more fully in the Examples that follow.

### EXAMPLES

### Example 1

### Method of Manufacture:

1050 grams of carbamazepine, 300 grams of silicified microcrystalline cellulose, 15 grams of sodium lauryl sulfate, all in powder form, were mixed in a suitable blender. The granulating dispersion containing 150 grams of ethyl cellulose, 375 grams of opadry [mixture of hydroxypropyl methyl cellulose mixed with polyethylene glycol] and 300 grams of water, was added to form a wet mass. The wet mass was then passed through a 0.8 mm screen using an extruder. The resulting rods (extrudate) were then placed on a spheronizer to produce spheres. These spheres were then dried using a fluid bed dryer until the moisture content was less than 2%. The composition of the pellets and the granulating solution used in making the pellet are summarized in Table 1A.

TABLE 1A

| | Quantity | |
|---|---|---|
| Ingredient | Grams/batch | % w/w |
| Ingredients in Powder form | | |
| Carbamazepine | 1050 | 72.92 |
| Silicified Microcrystalline Cellulose | 300 | 20.83 |
| Sodium Lauryl Sulfate | 15 | 1.04 |
| Granulating Solution | | |
| Ethyl cellulose latex dispersion as 25% w/w | 150 (37.5) | 2.6 |
| Hydroxypropyl methyl cellulose (Opadry) as | 375 (37.5) | 2.6 |
| 10% w/w dispersion | | |
| Water | 300 | — |

The dissolution of the pellets was carried out by dissolving the above-identified composition in pellet form in 1% Sodium Lauryl Sulfate Medium using a USP II apparatus at 50 rpm. The amount of carbamazepine that becomes dissolved in the SLS medium is determined at specific time intervals listed hereinbelow. The results are tabulated hereinbelow:

TABLE 1B

| Time | Cumulative % Released |
|---|---|
| (Hours) | in 1% SLS medium |
| 1 | 38.57 |
| 3 | 71.88 |
| 5 | 81.21 |
| 7 | 85.81 |

### Example 2

1050 grams of carbamazepine, 300 grams of silicified microcrystalline cellulose, 15 grams of sodium lauryl sulfate, and 37.5 grains of opadry, which are all the ingredients in the powder form, were mixed in a suitable blender. The granulating dispersion, containing 150 grams of ethyl cellulose in latex dispersion and 550 grams water, was added to form a wet mass. The wet mass was then passed through a 0.8 mm screen using an extruder. The resulting rods (extrudate) were then placed on a spheronizer to obtain spheres. These spheres were then dried using a fluid bed dryer until the moisture content was less than 2%. The composition of the pellets and the granulation solution utilized in preparing the pellets are summarized in Table 2A.

TABLE 2A

| | Quantity | |
|---|---|---|
| Ingredient | Grams/batch | % w/w |
| Ingredients in Powder form | | |
| Carbamazepine | 1050 | 72.92 |
| Silicified Microcrystalline Cellulose | 300 | 20.83 |
| Sodium Lauryl Sulfate | 15 | 1.04 |
| Opadry | 37.5 | 2.6 |
| Granulating Solution | | |
| Ethyl cellulose latex dispersion as 25% w/w | 150 (37.5) | 2.6 |
| Water | 550 | — |

Using the same procedure as in Example 1, the % of carbamazepine released was as follows:

TABLE 2B

| Time | Cumulative % Released |
|---|---|
| (Hours) | in 1% SLS medium |
| 1 | 30.62 |
| 3 | 52.82 |
| 5 | 65.17 |
| 7 | 68.62 |

### Example 3

1050 grams of carbamazepine, 300 grams of silicified microcrystalline cellulose, and 15 grams of sodium lauryl sulfate, which are all the ingredients in the powder form, were mixed in a suitable blender. The granulating dispersion, containing 150 grams of ethyl cellulose in latex dispersion, 375 grams of opadry, and 550 grams water, were added to form a wet mass. The wet mass was then passed through 1.0 mm screen using an extruder. The resulting rods (extrudate) were then placed on a spheronizer to produce spheres. These spheres were then dried using a fluid bed dryer until the moisture content was less than 2%. The composition of the pellet and the granulating solution used to make the pellet is summarized in Table 3A.

TABLE 3A

| | Quantity | |
|---|---|---|
| Ingredient | Grams/batch | % w/w |
| Ingredients in Powder form | | |
| Carbamazepine | 1050 | 72.92 |
| Silicified Microcrystalline Cellulose | 300 | 20.83 |
| Sodium Lauryl Sulfate | 15 | 1.04 |
| Granulating Solution | | |
| Ethyl cellulose latex dispersion as 25% w/w | 150 (37.5) | 2.6 |
| Opadry a 10% w/w dispersion | 375 (37.5) | 2.6 |
| Water | 550 | — |

Using the same procedure as in Example 1, the amount of drug released at various time intervals was as follows:

TABLE 3B

| Time | Cumulative % Released |
|---|---|
| (Hours) | in 1% SLS medium |
| 1 | 27.52 |
| 3 | 46.16 |
| 5 | 57.54 |
| 7 | 67.03 |

### Example 4

400 grams of mesalamine, 200 grams of silicified microcrystalline cellulose, and 76 grams of maltodextrin, which are all the ingredients in powder form, were mixed in a suitable blender. The granulating dispersion, containing 120 grams of ethyl cellulose in a latex dispersion and 40 grams opadry dispersion, was added to form a wet mass. The wet mass was then passed through a 1.0 mm screen using an extruder. The resulting rods (extrudate) were then placed on a spheronizer to produce spheres. These spheres were then dried using a fluid bed dryer until the moisture content was less than 2%. The composition of the pellets and the granulating solution used in making the pellet is summarized in Table 4A.

TABLE 4A

| | Quantity | |
|---|---|---|
| Ingredient | Grams/batch | % w/w |
| Ingredients in Powder form | | |
| Mesalamine | 400 | 56.33 |
| Silicified Microcrystalline Cellulose | 200 | 28.17 |
| Maltodextrin | 76 | 10.70 |
| Granulating Solution | | |
| Ethyl cellulose latex dispersion as 25% w/w | 120 (30) | 4.23 |
| Opadry a 10% w/w dispersion | 40 (4) | 0.56 |
| Water | — | — |

The dissolution of the pellets was carried out by dissolving the above-identified composition in an acetate buffer, pH 4.5, using a USP I apparatus at 100 rpm. The amount of drug that becomes dissolved is determined at specific time intervals tabulated hereinbelow.

TABLE 4B

| Time | Cumulative % Released |
|---|---|
| (Hours) | in pH 4.5 medium |
| 1 | 35 |
| 2 | 46 |
| 3 | 60 |
| 4 | 69 |
| 5 | 97 |
| 6 | 98 |

### Example 5

400 grams of mesalamine, 200 grams of silicified microcrystalline cellulose, 76 grams of maltodextrin, and 8 grams of opadry, which are all the ingredients in powder form, were mixed in a suitable blender. The granulating dispersion, containing 120 grams of ethyl cellulose, was added to form a wet mass. The wet mass was then passed through a 1.0 mm screen using an extruder. The resulting rods (extrudate) were then placed on a spheronizer to produce spheres. These spheres were then dried using a fluid bed dryer until the moisture content was less than 2%. The composition of the pellet and the granulating solution used to make the pellet are summarized in Table 5A.

TABLE 5A

| | Quantity | |
|---|---|---|
| Ingredient | Grams/batch | % w/w |
| Ingredients in Powder form | | |
| Mesalamine | 400 | 56.33 |
| Silicified Microcrystalline Cellulose | 200 | 28.17 |
| Maltodextrin | 72 | 10.14 |
| Opadry 29019 | 8 | 1.13 |
| Granulating Solution | | |
| Ethyl cellulose latex dispersion as 25% w/w | 120 (30) | 4.23 |
| Water | — | — |

The dissolution of the drug in solution was determined using the procedure described in Example 4. The results are tabulated below:

TABLE 5B

| Time | Cumulative % Released |
|---|---|
| (Hours) | in pH 4.5 medium |
| 1 | 42 |
| 2 | 57 |
| 3 | 66 |
| 4 | 72 |
| 5 | 76 |
| 6 | 82 |

### Examples 6-15

The pellets comprised of the active ingredient, propafenone hydrochloride, was prepared by mixing the various ingredients identified hereinbelow, using the methodology described in Example 1.

The dissolution of the composition was tested in pH 1.2 medium using USP apparatus II at 50 rpm. Dissolution results of the 325 mg capsules manufactured utilizing an extruder and spheronizer having the following composition are as follows:

| Ingredients | Batch 1 | Batch 2 | Batch 3 | Batch 4 |
|---|---|---|---|---|
| Propafenone | 325 mg | 325 mg | 325 mg | 325 mg |
| HCl | | | | |
| Polymer used | Eudragit NE | Acquacoat | Kollicoat SR | Surelease: |
| | 30 D: 25 mg | ECD 30: 25 mg | 30 D: 25 mg | 25 mg |
| Prosolv 50 | 10 mg | 10 mg | 10 mg | 10 mg |
| Malto M 180 | 5 mg | 5 mg | 5 mg | 5 mg |
| Water | 0.06 ml | 0.05 ml | 0.05 ml | 0.03 ml |
| Total weight | 365 mg | 365 mg | 365 mg | 365 mg |

50 rpm, paddle 1000 ml pH 1.2

| Batch 1 | Batch 2 | Batch 3 | Batch 4 |
|---|---|---|---|
| 15.75 | 14.48 | 14.30 | 8.33 |
| 27.13 | 26.39 | 23.49 | 17.82 |
| 44.59 | 41.31 | 37.12 | 30.31 |

| Ingredients | Batch 5 | Batch 6 | Batch 7 |
|---|---|---|---|
| Propafenone HCl | 325 mg | 325 mg | 325 mg |
| Polymer used | — | Kollicoat SR 30 D:15 mg | Surelease:Opadry |
| | | | 7006 clear |
| | | | (5 mg:5 mg) |
| PVPK 90 | — | 5 mg | — |
| Prosolv 50 | 20 | 13.3 mg | 20 mg |
| Malto M 180 | 20 | 6.67 mg | 10 mg |
| Water | 0.09 ml | 0.08 ml | 0.07 ml |
| Total weight | 365 mg | 365 mg | 365 mg |

50 rpm, paddle 1000 ml pH 1.2

| Time (hrs) | Batch 5 | Batch 6 | Batch 7 |
|---|---|---|---|
| 1 | 12.49 | 12.13 | 9.23 |
| 2 | 24.38 | 24.92 | 19.46 |
| 4 | 41.09 | 40.19 | 34.68 |
| 6 | 54.53 | 53.64 | 47.33 |
| 8 | 66.29 | 61.93 | 57.57 |
| 10 | 74.18 | 68.87 | 64.65 |
| 12 | 78.32 | 74.61 | 71.97 |

| Ingredients | Batch 8 | Batch 9 |
|---|---|---|
| Propafenone | 325 mg | 325 mg |
| HCl | | |
| Polymer used | Surelease 5 mg | Kollicoat SR 30D 5 mg |
| | i.e (20 mg | (i.e 16.66 mg |
| | dispersion) | dispersion) |
| Opadry 7006 | 10 mg | 10 mg |
| Prosolv 50 | 25 mg | 16.66 mg |
| Malto M 180 | — | 8.34 mg |
| Water | 0.086 ml | 0.067 ml |
| Total weight | 365 mg | 365 mg |

50 rpm, paddle 1000 ml pH 1.2

| Time (hrs) | Batch 8 | Batch 9 |
|---|---|---|
| 1 | 9.95 | 15.02 |
| 2 | 16.57 | 28.02 |
| 4 | 28.56 | 43.32 |
| 6 | 38.74 | 51.90 |
| 8 | 47.27 | 58.74 |
| 10 | 54.79 | 63.48 |
| 12 | 62.20 | 67.72 |

| Ingredients | Batch 10 | Batch 11 |
|---|---|---|
| Propafenone | 325 mg | 325 mg |
| HCl | | |
| Polymer used | Surelease 5 mg | Surelease 5 mg |
| Opadry 7006 | 5 mg | 5 mg |
| Tween (0.5%) | — | 1.825 |
| SLS (1%) | 3.65 mg | — |
| Prosolv 50 | 17.56 | |
| Malto M 180 | 8.79 | — |
| Water | 0.0733 ml | 0.066 ml |
| Total weight | 365 mg | 365 mg |

50 rpm, paddle 1000 ml pH 1.2

| Time (hrs) | Batch 10 | Batch 11 |
|---|---|---|
| 1 | 18.46 | 13.39 |
| 2 | 34.21 | 25.29 |
| 4 | 56.27 | 46.72 |
| 6 | 72.31 | 62.75 |
| 8 | 84.69 | 74.59 |
| 10 | 92.76 | 81.84 |
| 12 | 95.29 | 86.25 |

### Example 16

Using the procedures described in Example 5, the composition comprised of the following ingredients was prepared:

| Sr. No. | Ingredients | Qty in g per batch. | % w/w |
|---|---|---|---|
| | Ingredients in powder form | | |
| 1 | Mesalamine | 400 | 58.82 |
| 2 | Silicified Microcrystalline | 200 | 29.41 |
| | Cellulose | | |
| 3 | Maltodextrin M180 | 40 | 5.88 |
| | Granulating Solution | | |
| 4 | Ethyl cellulose latex | 160 (40) | 5.89 |
| | dispersion as 25% w/w | | |
| 5 | Water | — | — |
| | 1.0 mm screen used for | | |
| | extrusion | | |

The composition thus formed was placed in a fluid bed coating apparatus and was coated with the various coating dispersions described hereinbelow:

### A. Coating

| Sr. No. | Ingredients | Qty in mg per batch. | % w/w |
|---|---|---|---|
| 1 | Surelease | 13.5 | 90 |
| 2 | Opadry 7006 | 1.5 | 10 |
| 3 | Water | 85 | |

Although water was present in the coating dispersion, after the beads were coated, they were dried and as a consequence thereof, the water was evaporated.

The dissolution of the composition was determined using the same procedure as outlined in Example 4. The dissolution results were as follows:

900 ml medium, basket 100 rpm

| | 2% wt gain | | 3% wt gain | |
|---|---|---|---|---|
| Time | pH 1.2 buffer | pH 7.5 buffer | pH 1.2 buffer | pH 7.5 buffer |
| 1 h | 30.84 | 25.27 | 14.97 | 15.83 |
| 2 | 44.27 | 40.52 | 22.83 | 23.97 |
| 3 | 55.56 | 52.13 | 30.52 | 31.42 |
| 4 | 64.03 | 57.46 | 35.85 | 36.87 |
| 5 | 69.96 | 68.04 | 39.68 | 40.30 |

### B. Coating

The composition was coated as described above with the following composition and dried.

| Sr. No. | Ingredients | Qty in mg per batch. | % w/w |
|---|---|---|---|
| 1 | Opadry OY-S- | 3.3 | 100 |
| | 29019 clear | | |
| 2 | Water | 96.7 | |

The dissolution was determined as described above. The results were as follows:

900 ml medium, basket 100 rpm

| | 3% wt gain | |
|---|---|---|
| Time | PH 1.2 buffer | pH 7.5 buffer |
| 1 h | 67.99 | 58.75 |
| 2 | 86.99 | 77.41 |
| 3 | 97.89 | 91.41 |
| 4 | 99.32 | 93.62 |

### Example 17

Using the procedure of Example 5, uncoated mesalamine beads containing the following ingredients were prepared:

| Sr. | | Qty in g | |
|---|---|---|---|
| No. | Ingredients | per Batch | % w/w |
| | Ingredients in powder form | | |
| 1 | Mesalamine | 800 | 58.82 |
| 2 | Silicified Microcrystalline Cellulose | 400 | 29.41 |
| 3 | Maltodextrin M180 | 80 | 5.88 |
| | Granulating Solution | | |
| 4 | Ethyl cellulose latex dispersion | 320 (80) | 5.89 |
| | as 25% w/w | | |
| 5 | Water | 1600 g | |
| | 1.0 mm screen used for extrusion | | |

Using the coating procedure described in Example 16, the beads herein were coated with the following composition:

| Sr. | | Qty in mg | |
|---|---|---|---|
| No. | Ingredients | per batch. | % w/w |
| 1 | Surelease | 11.25 | 75 |
| 2 | Sureteric | 3 | 20 |
| 3 | Opadry 7006 | 0.75 | 5 |
| 4 | Water | 85 | — |

The dissolution of the coated beads was determined as described in Example 16. The results were as follows:

900 ml medium, basket 100 rpm

| | 3% wt gain | |
|---|---|---|
| Time | pH 1.2 buffer | pH 7.5 buffer |
| 1 | 30.02 | 37.29 |
| 2 | 40.67 | 55.63 |
| 3 | 50.36 | 66.80 |
| 4 | 57.55 | 75.19 |
| 5 | 63.98 | 80.89 |

### Example 18

Using the procedure of Example 5, the following uncoated mesalamine beads were prepared:

| Sr. | | Qty in g | |
|---|---|---|---|
| No. | Ingredients | per Batch | % w/w |
| | Ingredients in powder form | | |
| 1 | Mesalamine | 1000 | 58.82 |
| 2 | Silicified Microcrystalline Cellulose | 500 | 29.41 |
| 3 | Maltodextrin M180 | 100 | 5.88 |
| | Granulating Solution | | |
| 4 | Ethyl cellulose latex dispersion | 400 (100) | 5.89 |
| | as 25% w/w | | |
| 5 | Water | | |
| | 1.0 mm screen used for extrusion | | |

Using the coating procedure described in Example 16, the beads were coated with one of the following compositions and dried.

### A. Coating

| Sr. | | Qty in mg | |
|---|---|---|---|
| No. | Ingredients | per batch. | % w/w |
| 1 | Surelease | 13.5 | 90 |
| 2 | Opadry ys-1-29019 | 1.5 | 10 |
| 3 | Water | 85 | |

The dissolution of the coated composition was determined as described in Example 16. The results were as follows:

900 ml medium, basket 100 rpm

| | 2% wt gain | | |
|---|---|---|---|
| Time | pH 1.2 buffer | pH 4.5 buffer | pH 7.5 buffer |
| 1 h | 27.98 | 6.93 | 45.60 |
| 2 | 50.57 | 10.34 | 70.86 |
| 3 | 65.74 | 13.64 | — |
| 4 | 86.33 | 18.69 | 92.5 |
| 5 | — | — | |
| 6 | 93.32 | 21.48 | — |

### B. Coating

| Sr. | | Qty in mg | |
|---|---|---|---|
| No. | Ingredients | per batch. | % w/w |
| 1 | Surelease | 13.5 | 90 |
| 2 | Opadry ys-1-7006 | 1.5 | 10 |
| 3 | Water | 172.5 | |

The dissolution profile of the coating composition was determined using the procedure of Example 16. The results were as follows:

900 ml medium, basket 100 rpm

| | 1.5% wt gain | | 2% wt gain | |
|---|---|---|---|---|
| | pH 1.2 | pH 4.5 | pH 1.2 | pH 7.5 |
| Time | buffer | buffer | buffer | buffer. |
| l h | 21.85 | 5.61 | 12.52 | 18.43 |
| 2 | 38.29 | 8.71 | 24.19 | 32.48 |
| 3 | 52.78 | 12.0 | 32.66 | — |
| 4 | 61.05 | 14.10 | 40.95 | 52.92 |
| 5 | 77.02 | 18.53 | — | — |

### C. Coating

| Sr. | | Qty in mg | |
|---|---|---|---|
| No. | Ingredients | per batch. | % w/w |
| 1 | Surelease | 12 | 80 |
| 2 | Opadry ys-1-7006 | 3 | 20 |
| 3 | Water | 135 | |

The dissolution profile of this composition was determined as described in Example 16. The results were as follows:

900 ml medium, basket 100 rpm

| | 1% wt gain | | | 2% wt gain | | |
|---|---|---|---|---|---|---|
| | pH 1.2 | pH 4.5 | pH 7.5 | pH 1.2 | pH 4.5 | pH 7.5 |
| Time | buffer | buffer | buffer | buffer | buffer | buffer |
| 1 h | 37.55 | 10.24 | 40.59 | 20.94 | 8.71 | 21.84 |
| 2 | 59.97 | 16.22 | 59.73 | 39.97 | 13.41 | 35.17 |
| 3 | 74.54 | 20.58 | 72.24 | 52.89 | 17.41 | 57.74 |
| 4 | 83.30 | 24.50 | 81.42 | 59.01 | 21.91 | 68.50 |
| 5 | 87.92 | 27.80 | 90.10 | — | 25.62 | 76.95 |
| 6 | — | — | — | — | 27.98 | 83.15 |

### Example 19

Using the procedure of Example 5, uncoated mesalamine beads were prepared containing the following ingredients:

| Sr. | | Qty in g | |
|---|---|---|---|
| No. | Ingredients | per batch. | % w/w |
| | Ingredients in powder form | | |
| 1 | Mesalamine | 1000 | 58.52 |
| 2 | Silicified Microcrystalline Cellulose | 500 | 29.41 |
| 3 | Maltodextrin | 100 | 5.88 |
| | Granulating Solution | | |
| 4 | Ethyl cellulose latex dispersion | 400 (100) | 5.89 |
| | as 25% w/w | | |
| 5 | Water | — | |
| | 1.0 mm screen used for extrusion | | |

Using the procedure of Example 16, they were coated using the procedure described above with the following dispersion and dried.

| Sr. | | Qty in mg | |
|---|---|---|---|
| No. | Ingredients | per batch. | % w/w |
| 1 | Surelease | 12 | 80 |
| 2 | Opadry ys-1-7006 | 3 | 20 |
| 3 | Water | 85 | |

The dissolution profile was determined, as described in Example 16. The results were as follows:

900 ml medium, basket 100 rpm

| | PH 1.2 buffer | | | | pH 7.5 buffer | | | | pH 4.5 buffer | |
|---|---|---|---|---|---|---|---|---|---|---|
| Time | 0.5% | 0.75% | 1% | 2% | 0.5% | 0.75% | 1% | 2% | 1% | 2% |
| 1 h | 57.51 | 45.65 | 34.36 | 11.37 | 54.25 | 48.98 | 37.55 | 18.68 | 9.44 | 4.90 |
| 2 | 79.26 | 68.0 | 54.87 | 20.49 | 74.42 | 70.29 | 57.35 | 31.36 | 14.17 | 6.24 |
| 3 | 89.79 | 81.19 | 68.32 | 27.51 | 85.97 | 83.61 | 70.71 | 42.26 | 18.18 | 7.84 |
| 4 | 95.19 | 89.19 | 77.74 | 34.20 | 93.66 | 88.71 | 79.16 | 51.38 | 21.38 | 9.08 |
| 5 | — | — | 84.15 | 40.14 | — | — | 88.84 | 59.64 | 25.04 | 10.51 |

### Example 20

Using the procedure of Example 5, the mesalamine beads were prepared containing the following ingredients:

| Sr. | | Qty in g | |
|---|---|---|---|
| No. | Ingredients | per Batch | % w/w |
| | Ingredients in powder form | | |
| 1 | Mesalamine | 800 | 56.34 |
| 2 | Silicified Microcrystalline Cellulose | 400 | 28.17 |
| 3 | Maltodextrin | 144 | 10.14 |
| 4 | Opadry 7006 | 16 | 1.13 |
| | Granulating Solution | | |
| 5 | Ethyl cellulose latex dispersion | 240 (60) | 4.22 |
| | as 25% w/w | | |
| 6 | Water | — | |
| | 1.0 mm screen used for extrusion | | |

The uncoated beads were coated with the following dispersion and dried using the procedure in Example 16

| Sr. | | Qty in mg | |
|---|---|---|---|
| No. | Ingredients | per batch. | % w/w |
| 1 | Surelease | 12 | 80 |
| 2 | Opadry | 3 | 20 |
| 3 | Water | 135 | |

The dissolution of this composition was determined using the procedure of Example 16. The results were as follows:

900 ml medium, basket 100 rpm

| | pH 1.2 buffer | | | pH 7.5 buffer | | | pH 4.5 buffer | | |
|---|---|---|---|---|---|---|---|---|---|
| Time | 1% | 1.5% | 2% | 1% | 1.5% | 2% | 1% | 1.5% | 2% |
| 1 h | 71.02 | 57.10 | 41.15 | 72.43 | 63.17 | 57.98 | 33.06 | 26.55 | 19.33 |
| 2 | 89.07 | 77.87 | 59.03 | 89.85 | 81.61 | 76.62 | 44.83 | 37.09 | 25.96 |
| 3 | 95.29 | 91.65 | 72.04 | 97.68 | 95.32 | 88.20 | 54.86 | 45.97 | 32.66 |
| 4 | 97.39 | 94.21 | 80.93 | 100.30 | 98.52 | 90.04 | 62.06 | 53.33 | 38.46 |
| 5 | — | — | 85.41 | — | — | 96.30 | 67.27 | 58.72 | 43.33 |
| 6 | — | — | — | — | — | — | 72.45 | 63.90 | 47.63 |

### Example 21

Using the procedure described in Example 5, the uncoated mesalamine beads were prepared containing the following ingredients:

| Sr. | | Qty in g | |
|---|---|---|---|
| No. | Ingredients | per Batch | % w/w |
| | Ingredients in powder form | | |
| 1 | Mesalamine | 800 | 56.34 |
| 2 | Silicified Microcrystalline Cellulose | 400 | 28.17 |
| 3 | Maltodextrin | 144 | 10.14 |
| 4 | Opadry 29019 | 16 | 1.13 |
| | Granulating Solution | | |
| 5 | Ethyl cellulose latex dispersion | 240 (60) | 4.22 |
| | as 25% w/w | | |
| 6 | Water | 40 | |
| | 1.0 mm screen used for extrusion | | |

The uncoated beads were coated with the following dispersion as described in Example 16 and then dried.

| Sr. No. | Ingredients | Qty in mg per batch. | % w/w |
|---|---|---|---|
| 1 | Surelease | 12 | 80 |
| 2 | Opadry | 3 | 20 |
| 3 | Water | 135 | |

The dissolution of this coated composition was determined using the procedure of Example 16. The results were as follows:

900 ml medium, basket 100 rpm

| | pH 1.2 buffer | | | pH 7.5 buffer | | | pH 4.5 buffer | | |
|---|---|---|---|---|---|---|---|---|---|
| Time | 1% | 1.5% | 2% | 1% | 1.5% | 2% | 1% | 1.5% | 2% |
| 1 | 58.09 | 32.80 | 18.00 | 57.57 | 44.91 | 43.44 | 22.81 | 12.38 | 9.17 |
| 2 | 81.23 | 53.21 | 31.13 | 77.82 | 57.57 | 64.86 | 34.47 | 19.65 | 12.13 |
| 3 | 89.25 | 67.22 | 42.76 | 87.94 | 78.15 | 80.58 | 43.85 | 24.41 | 15.20 |
| 4 | 95.95 | 87.24 | 53.86 | 90.24 | 89.04 | 89.65 | 51.01 | 28.78 | 18.04 |
| 5 | — | — | 61.65 | — | 93.99 | 94.52 | — | 33.37 | 20.64 |
| 6 | — | — | — | — | | | — | 36.40 | 23.45 |

### Example 22

The uncoated mesalamine beads were prepared in accordance with the procedure of Example 5 using the following ingredients:

| Sr. No. | Ingredients | Qty in g per Batch | % w/w |
|---|---|---|---|
| | Ingredients in powder form | | |
| 1 | Mesalamine | 400 | 50.00 |
| 2 | Silicified Microcrystalline | 236 | 29.5 |
| | Cellulose | | |
| 3 | Maltodextrin | 80 | 10.00 |
| | Granulating Solution | | |
| 4 | Ethyl cellulose latex | 320(80) | 10.00 |
| | dispersion as 25% w/w | | |
| 5 | Opadry 7006 | 40(4) | 0.50 |
| 6 | Water | 110 | |
| | 1.0 mm screen used | | |
| | for extrusion | | |

The uncoated beads were coated with the following dispersion using the procedure of Example 16 and then dried.

| Sr. No. | Ingredients | Qty in mg per batch. | % w/w |
|---|---|---|---|
| 1 | Surelease | 12 | 80 |
| 2 | Opadry | 3 | 20 |
| 3 | Water | 135 | |

The dissolution of this coating composition was determined as described in Example 16. The results were as follows:

900 ml medium, basket 100 rpm

| | PH 1.2 buffer | | | pH 7.5 buffer | | |
|---|---|---|---|---|---|---|
| Time | 1% | 1.5% | 2% | 1% | 1.5% | 2% |
| 1 h | 87.79 | 73.15 | 64.31 | 83.59 | 71.74 | 71.91 |
| 2 | 97.52 | 90.81 | 86.79 | 99.76 | 91.40 | 93.31 |
| 3 | 99.41 | 94.84 | 92.93 | — | 97.86 | 99.36 |
| 4 | — | 95.71 | 94.73 | — | — | — |

### Example 23

2 sets of uncoated mesalamine beads were prepared using the procedure of Example 5, described herein.

Batch #1

| Sr. No. | Ingredients | Qty in g per Batch | % w/w |
|---|---|---|---|
| | Ingredients in powder form | | |
| 1 | Mesalamine | 400 | 50.00 |
| 2 | Silicified Microcrystalline | 236 | 29.5 |
| | Cellulose | | |
| 3 | Maltodextrin M180 | 80 | 10.00 |
| | Granulating Solution | | |
| 4 | Ethyl cellulose latex dispersion | 320(80) | 10.00 |
| | as 25% w/w | | |
| 5 | Opadry 7006 | 40(4) | 0.50 |
| 6 | Water | 140 | |
| | 1.0 mm screen used for | | |
| | extrusion | | |

Batch No. 2

| Sr. No. | Ingredients | Qty in g per Batch | % w/w |
|---|---|---|---|
| | Ingredients in powder form | | |
| 1 | Mesalamine | 400 | 50.00 |
| 2 | Silicified Microcrystalline | 236 | 29.5 |
| | Cellulose | | |
| 3 | Maltodextrin M180 | 80 | 10.00 |
| | Granulating Solution | | |
| 4 | Ethyl cellulose latex dispersion | 320(80) | 10.00 |
| | as 25% w/w | | |
| 5 | Opadry 7006 | 40(4) | 0.50 |
| 6 | Water | 160 | |
| | 1.0 mm screen used | | |
| | for extrusion | | |

Both the batches were combined and coated with the following ingredients using the procedure of Example 16:

| Sr. No. | Ingredients | Qty in mg per batch | % w/w |
|---|---|---|---|
| 1 | Surelease | 12 | 80 |
| 2 | Opadry | 3 | 20 |
| 3 | Water | 135 | |

The dissolution of the combined composition was determined as described in Example 16. The results were as follows:

900 ml medium, basket 100 rpm

| | pH 1.2 buffer | | | pH 7.5 buffer | | |
|---|---|---|---|---|---|---|
| Time | 1% | 1.5% | 2% | 1% | 1.5% | 2% |
| l h | 79.69 | 72.90 | 70.28 | 76.85 | 69.92 | 71.48 |
| 2 | 93.83 | 91.38 | 91.60 | 94.23 | 90.26 | 93.56 |
| 3 | 95.95 | 96.56 | 97.03 | 98.99 | 100.43 | 102.56 |
| 4 | 97.87 | 96.39 | — | 100.15 | — | 104.80 |
| | 98.19 | — | — | — | — | — |

### Example 24

The uncoated mesalamine beads were prepared in accordance with the procedure of Example 5 containing the following ingredients:

| Sr. No. | Ingredients | Qty in g per Batch | % w/w |
|---|---|---|---|
| | Ingredients in powder form | | |
| 1 | Mesalamine | 400 | 50.00 |
| 2 | Silicified Microcrystalline | 236 | 29.5 |
| | Cellulose | | |
| 3 | Maltodextrin M180 | 80 | 10.00 |
| | Granulating Solution | | |
| 4 | Ethyl cellulose latex dispersion | 320(80) | 10.00 |
| | as 25% w/w | | |
| 5 | Opadry 7006 | 40(4) | 0.50 |
| 6 | Water | 144 | |
| | 1.0 mm screen used | | |
| | for extrusion | | |

Following the procedures of Example 16, the beads were coated with one of the compositions described hereinabove and dried.

### A. Coating

| Sr. No. | Ingredients | Qty in mg per batch. | % w/w |
|---|---|---|---|
| 1 | Surelease | 12 | 80 |
| 2 | Opadry | 3 | 20 |
| 3 | Water | 135 | |

The dissolution profile was determined using procedure of Example 16. The results were as follows

900 ml medium, basket 100 rpm

| | Ph 1.2 buffer | | | pH 7.5 buffer | | pH 4.5 buffer |
|---|---|---|---|---|---|---|
| Time | 2% | 2.5% | 3% | 2% | 2.5% | 3% |
| l h | 61.61 | 54.57 | 51.95 | 89.65 | 81.52 | 70.62 |
| 2 | 87.33 | 79.23 | 75.28 | 101.12 | 97.74 | 92.17 |
| 3 | 95.25 | 89.76 | 87.64 | — | 102.72 | 100.02 |
| 4 | 97.76 | 94.42 | 94.08 | — | — | — |

### B. Coating

| Sr. No. | Ingredients | Qty in mg per batch. | % w/w |
|---|---|---|---|
| 1 | Surelease | 13.5 | 90 |
| 2 | Opadry | 1.5 | 10 |
| 3 | Water | 135 | |

The dissolution of this composition was determined using the procedure described in Example 16. The results were as follows

900 ml medium, basket 100 rpm

| | pH 1.2 buffer | | | pH 7.5 buffer | | | pH 4.5 buffer | |
|---|---|---|---|---|---|---|---|---|
| Time | 2% | 2.5% | 3% | 2% | 2.5% | 3% | 2.5% | 3% |
| l h | 53.84 | 41.15 | 26.26 | 70.09 | 62.05 | 54.78 | 16.13 | 8.38 |
| 2 | 76.36 | 62.23 | 43.49 | 90.43 | 84.63 | 77.37 | 24.42 | 13.19 |
| 3 | 86.29 | 74.62 | 55.26 | 97.92 | 94.22 | 89.13 | 30.30 | 17.35 |
| 4 | 92.22 | 81.73 | 64.70 | — | — | 95.15 | 35.44 | 21.19 |
| 5 | 94.98 | 87.04 | 79.73 | — | — | 96.67 | 38.59 | 24.27 |
| 6 | 97.34 | 91.08 | — | — | — | — | 44.71 | 30.02 |
| 7 | — | — | — | — | — | — | 48.40 | 32.75 |

### Examples 25-30

The following beads/pellets were prepared in accordance with the procedure described hereinabove. In addition, the dissolution of these beads/pellets is provided hereinbelow. The dissolution profile was determined using the procedure described in Example 16.

### Example 25

| | Quantity | |
|---|---|---|
| Ingredient | Mg/Capsule | % w/w |
| Ingredients in Powder form | | |
| Propafenone | 325 | |
| Prosolve 50 | 13.3 | |
| Maltodextrin | 6.67 | |
| Granulating Solution | | |
| Kollicoat SR 30 D (@ 30% w/w dispersion) | 15 | |
| PVP K90 | 5 | |
| Water | 0.08 | |
| 1.0 mm screen used for extrusion | | |

1000 ml medium, Paddle 50 rpm

| Time (Hours) | Cumulative % Released in pH 1.2 medium |
|---|---|
| 1 | 12.13 |
| 2 | 24.92 |
| 4 | 40.19 |
| 6 | 53.64 |
| 8 | 61.93 |
| 10 | 68.87 |
| 12 | 74.61 |

### Example 26

| | Quantity | |
|---|---|---|
| Ingredient | Mg/Capsule | % w/w |
| Ingredients in Powder form | | |
| Propafenone | 325 | |
| Prosolve 50 | 20 | |
| Maltodextrin | 10 | |
| Granulating Solution | | |
| Surelease (@ 25% w/w dispersion) | 5 | |
| Opadry | 5 | |
| Water | 0.07 | |
| 1.0 mm screen used for extrusion | | |

1000 ml medium, Paddle 50 rpm

| Time (Hours) | Cumulative % Released in pH 1.2 medium |
|---|---|
| 1 | 9.23 |
| 2 | 19.46 |
| 4 | 34.68 |
| 6 | 47.33 |
| 8 | 57.57 |
| 10 | 64.65 |
| 12 | 71.97 |

### Example 27

| | Quantity | |
|---|---|---|
| Ingredient | Mg/Capsule | % w/w |
| Ingredients in Powder form | | |
| Propafenone | 325 | |
| Prosolve 50 | 25 | |
| Granulating Solution | | |
| Surelease (@ 25% w/w dispersion) | 5 | |
| Opadry | 10 | |
| Water | 0.086 | |
| 1.0 mm screen used for extrusion | | |

1000 ml medium, Paddle 50 rpm

| Time (Hours) | Cumulative % Released in pH 1.2 medium |
|---|---|
| 1 | 9.95 |
| 2 | 16.57 |
| 4 | 28.56 |
| 6 | 38.74 |
| 8 | 47.27 |
| 10 | 54.79 |
| 12 | 62.20 |

### Example 28

| | Quantity | |
|---|---|---|
| Ingredient | Mg/Capsule | % w/w |
| Ingredients in Powder form | | |
| Propafenone | 325 | |
| Prosolve 50 | 16.66 | |
| Maltodextrin | 8.34 | |
| Granulating Solution | | |
| Kollicoat SR 30 D (@ 30% w/w dispersion) | 5 | |
| Opadry 7006 | 10 | |
| Water | 0.067 | |
| 1.0 mm screen used for extrusion | | |

1000 ml medium, Paddle 50 rpm

| Time (Hours) | Cumulative % Released in pH 1.2 medium |
|---|---|
| 1 | 15.02 |
| 2 | 28.02 |
| 4 | 43.32 |
| 6 | 51.90 |
| 8 | 58.74 |
| 10 | 63.48 |
| 12 | 67.72 |

### Example 29

| | Quantity | |
|---|---|---|
| Ingredient | Mg/Capsule | % w/w |
| Ingredients in Powder form | | |
| Propafenone | 325 | |
| Prosolve 50 | 17.56 | |
| Maltodextrin | 8.79 | |
| SLS | 3.65 | |
| Granulating Solution | | |
| Surelease (@ 25% w/w dispersion) | 5 | |
| Opadry 7006 | 5 | |
| Water | 0.0733 | |
| 1.0 mm screen used for extrusion | | |

1000 ml medium, Paddle 50 rpm

| Time (Hours) | Cumulative % Released in pH 1.2 medium |
|---|---|
| 1 | 18.46 |
| 2 | 34.21 |
| 4 | 56.27 |
| 6 | 72.31 |
| 8 | 84.69 |
| 10 | 92.76 |
| 12 | 95.29 |

### Example 30

| | Quantity | |
|---|---|---|
| Ingredient | Mg/Capsule | % w/w |
| Ingredients in Powder form | | |
| Propafenone | 325 | |
| Prosolve 50 | 17.56 | |
| Maltodextrin | 8.79 | |
| Tween 80 | 1.825 | |
| Granulating Solution | | |
| Surelease (@ 25% w/w dispersion) | 5 | |
| Opadry 7006 | 5 | |
| Water | 0.066 | |
| 1.0 mm screen used for extrusion | | |

1000 ml medium, Paddle 50 rpm

| Time (Hours) | Cumulative % Released in pH 1.2 medium |
|---|---|
| 1 | 13.39 |
| 2 | 25.29 |
| 4 | 46.72 |
| 6 | 62.75 |
| 8 | 74.59 |
| 10 | 81.84 |
| 12 | 86.25 |

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

What is claimed is:

1. A sustained release pharmaceutical composition in pellet form, the core of said pellet comprising:
(a) a therapeutically effective amount of a medicament;
(b) 0.5 to 50% by weight of a water-soluble polymer;
(c) 1 to 25% by weight of a water-insoluble polymer applied in an aqueous latex dispersion and subsequently the water removed; and wherein the sum of the percentages of the medicament, the water-insoluble polymer and the water-soluble polymer is equal to or less than 100%.

2. The sustained release pharmaceutical composition according to claim 1, wherein the weight percentage of the water-soluble polymer ranges from about 1 to about 20% by weight of said composition.

3. The sustained release pharmaceutical composition according to claim 2, wherein the weight percentage of the water-soluble polymer ranges from about 2 to about 10% by weight of same composition.

4. The sustained release pharmaceutical composition according to claim 1, wherein the weight percentage of the water-insoluble polymer ranges from about 3 to about 20% by weight of said composition.

5. The sustained release pharmaceutical composition according to claim 1, wherein the weight percentage of the water-insoluble polymer ranges from about 5 to about 15% by weight of said composition.

6. The sustained release pharmaceutical composition according to claim 1, wherein the ratio of the water-soluble polymer to the water-insoluble polymer ranges from about 5:95 to about 50:50.

7. The sustained release pharmaceutical composition according to claim 6, wherein the ratio of the water-soluble polymer to the water-insoluble polymer ranges from about 5:95 to about 30:70.

8. The sustained release pharmaceutical composition according to claim 1, wherein the ratio of the water-soluble polymer to the water-insoluble polymer ranges from about 5:95 to about 20:80.

9. The sustained release pharmaceutical composition according to claim 1, wherein the water-soluble polymer is a cellulose derivative selected from the group consisting of hydroxypropyl methylcellulose (HPMC), hydroxypropyl cellulose (HPC), sodium carboxymethylcellulose (NaCMC), hydrocolloid, polyvinyl alcohol (PVA) and polyvinylpyrrolidone (PVP).

10. The sustained release pharmaceutical composition according to claim 9, wherein the water-soluble polymer is hydroxypropyl methylcellulose.

11. The sustained release pharmaceutical composition according to claim 1, wherein the water-insoluble polymer is a cellulose derivative selected from the group consisting of ethyl cellulose and cellulose acetate, and methylmethacrylate copolymers.

12. The sustained release pharmaceutical composition according to claim 11, wherein the water-insoluble polymer is ethyl cellulose.

13. The sustained release pharmaceutical composition according to claim 1, further comprising a diluent, wherein the diluent is present in the core in an amount ranging from about 5% to about 95% by weight of the total composition.

14. The sustained release pharmaceutical composition according to claim 13, wherein the diluent is present in the core in an amount which ranges from about 10% to about 60% by weight of the total composition.

15. The sustained release pharmaceutical composition according to claim 14, wherein the diluent is present in the core in an amount which ranges from about 20% to about 40% by weight of the total composition.

16. The sustained release pharmaceutical composition according to claim 13, wherein the diluent is selected from the group consisting of microcrystalline cellulose, starch and maltodextrin.

17. The sustained release pharmaceutical composition according to claim 13, wherein the diluent is microcrystalline cellulose or maltodextrin.

18. The sustained release pharmaceutical composition according to claim 1, further comprising a surfactant in the core ranging from about 0.1 to about 5% by weight of the total composition.

19. The sustained release pharmaceutical composition according to claim 18, further comprising a surfactant ranging from about 0.5 to about 2% by weight of the total composition.

20. The sustained release pharmaceutical composition according to claim 18, wherein the surfactant is sodium lauryl sulfate.

21. The sustained release pharmaceutical composition according to claim 19, wherein the surfactant is sodium lauryl sulfate.

22. The sustained release pharmaceutical composition according to claim 1, wherein the diameter of the pellet ranges from about 0.2 mm to about 2.5 mm.

23. The sustained release pharmaceutical composition according to claim 1, wherein the diameter of the pellet ranges from about 0.5 mm to about 1.5 mm.

24. The sustained release pharmaceutical composition according to claim 1, wherein the diameter of the pellet ranges from about 0.8 mm to about 1.2 mm.

25. A method of preparing a sustained release pharmaceutical composition in pellet form wherein the core is prepared by the steps comprising:
(a) blending a therapeutically effective amount of a medicament; and 0.5 to 50% by weight of a water-soluble polymer;
(b) adding a granulating liquid to form a wet mass, wherein the granulating liquid comprises 1 to 25% by weight of a water-insoluble polymer applied as an aqueous latex dispersion and the water subsequently removed, and wherein the sum of the percentages of the medicament, the water-insoluble polymer and the water-insoluble polymer is equal to or less than 100%
(c) passing the wet mass through a perforated screen to obtain an extrudate; and
(d) forming spheres from the extrudate.

26. The method according to claim 25, wherein the extrudate in step (d) are placed on a spinning plate to make spherical pellets.

27. The method according to claim 25, further comprising drying the spherical pellets by fluid bed drying or tray drying.

28. The method according to claim 27, wherein the spherical pellets are dried by fluid bed drying.

29. A method of preparing a sustained release pharmaceutical composition in pellet form, wherein the core of the pellet is prepared by the steps comprising:
(a) blending a therapeutically effective amount of a medicament and 0.5 to 50% by weight of a water-soluble polymer;
(b) adding a granulating liquid to form a wet mass, wherein the granulating liquid comprises 1 to 25% by weight of a water-insoluble polymer applied as an aqueous latex dispersion and subsequent thereto, the water is removed, and wherein the sum of the percentages of the medicament, the water-insoluble polymer and the water-soluble polymer is equal to or less than 100%; and
(c) placing the wet mass from step (b) onto a rotor processor to form spheres wherein the granulating liquid is sprayed onto the blend.

30. The method according to claim 29, further comprising drying the spherical pellets by fluid bed drying or tray drying.

31. The method according to claim 30, wherein the spherical pellets are dried by fluid bed drying.

32. A method of treating a disease in a patient requiring a medicament for treating said disease, said treatment comprising administering to the patient an effective amount of the sustained release pharmaceutical composition of claim 1.

33. The method according to claim 32 wherein the water-insoluble polymer is present in the core of the pellet in an amount ranging from about 2% to about 25% by weight of the pharmaceutical composition.

34. The composition according to claim 1, wherein said active medicament is carbamazepine.

35. The composition according to claim 1, wherein said active medicament is mesalamine.

36. The composition according to claim 1, wherein said active medicament is propafenone.

37. The method according to claim 25, wherein the water-soluble polymer is dissolved in the aqueous latex dispersion of the water-insoluble polymer.

38. The method according to claim 25, wherein the water-soluble polymer is added as a powder as well as dissolved in the aqueous latex dispersion.

39. The compositions according to claim 1, wherein the medicament has a water solubility of less than about 40 mg/ml.

40. The compositions according to claim 1, wherein the medicament has a water solubility of less than about 25 mg/ml.

41. The compositions according to claim 1, wherein the medicament has a water solubility of less than about 1 mg/ml.
